(19) United States
(12) Patent Application Publication
(10) Pub. No.: US 2016/0271035 A1
(43) Pub. Date: Sep. 22, 2016

(54) ODOR-REDUCED AGENT FOR SMOOTHING OR PERMANENTLY SHAPING HAIR OR FOR DEPILATION

(71) Applicant: Henkel AG & Co. KGaA, Duesseldorf (DE)

(72) Inventors: Norbert Schettiger, Hilden (DE); Constanze Neuba, Grevenbroich (DE); Frank Janssen, Koeln (DE); Burkhard Mueller, Duesseldorf (DE)

(73) Assignee: Henkel AG & Co. KGaA, Duessledorf (DE)

(21) Appl. No.: 15/168,869

(22) Filed: May 31, 2016

Related U.S. Application Data
(63) Continuation of application No. PCT/EP2014/076178, filed on Dec. 2, 2014.

(30) Foreign Application Priority Data
Dec. 17, 2013 (DE) 10 2013 226 278.7

(51) Int. Cl. A61K 8/46; A61Q 9/04; A61K 8/37; A45D 2/02; A61K 8/86; A45D 7/04; A45D 2/12; A61Q 5/04; A61K 8/34

(52) U.S. Cl. CPC A61K 8/46; A61Q 5/04; A61Q 9/04; A61K 8/375; A61K 8/342; A61K 8/86; A45D 7/045; A45D 2/127; A45D 2/02; A61K 2800/884

(57) ABSTRACT

An agent for straightening, permanently shaping, or depilation of keratinous fibers comprising, (a) at least one linear alkan-1-ol or alken-1-ol in each case having 20-22 carbon atoms, wherein the at least one alken-1-ol has one to four olefinic double bonds, in a total quantity of 1.8 to 10.0% by weight, and (b) at least one glyceryl fatty acid ester of general formula (I), wherein R1, R2, and R3 independently stand for a hydrogen atom or a group of formula (II), R4 stands for an unbranched or branched, saturated or unsaturated Cu₁₁-C₂₇ alkyl group, with the condition that at least one and at most two of the moieties selected from R1, R2, and R3 is a group of formula (II), (c) water, and (d) at least one keratin-reducing compound selected from thioglycolic acid or salts thereof, thiolactic acids or salts thereof, cysteine, acetylcysteine, cysteamine, or salts thereof, and mixtures thereof.

### FIELD OF THE INVENTION

The present invention generally relates to agents for straightening, permanently shaping, or depilation of keratinous fibers, in particular human hair, which minimize the intense, irritating odor of the sulfur-containing reducing agents (thioglycolate, cysteine, etc.) which occurs with permanent wave, hair straightening, and depilatory agents.

### BACKGROUND OF THE INVENTION

The permanent deformation of keratin fibers is usually carried out in such a way that the fiber is deformed with the aid of mechanical deformation means (curlers, papillotes), and the deformation is fixed by suitable auxiliary agents. Before and/or after this deformation, the fiber is treated with the aqueous preparation of a keratin-reducing substance, and after an exposure period is rinsed with water or an aqueous solution. In a second step, the fiber is then treated with the aqueous preparation of an oxidizing agent. After an exposure period, the oxidizing agent is also rinsed out, and the mechanical deformation means (curlers, papillotes) are removed from the fiber.

The aqueous preparation of the keratin-reducing substance is usually set alkaline so that on the one hand a sufficient portion of the thiol functions is present in deprotonated form, and on the other hand the fiber swells, thus allowing deep penetration of the keratin-reducing substance into the fiber. The keratin-reducing substance cleaves a portion of the disulfide bonds of the keratin to form —SH groups, resulting in loosening of the peptide interlinkage, and, due to the tension on the fiber from the mechanical deformation, a reorientation of the keratin structure. Disulfide bonds are relinked under the influence of the oxidizing agent, thus refixing the keratin structure in the predefined deformation. The permanent wave treatment of human hair represents a known method of this type. This method may be used for creating curls and waves in straight hair, and also for straightening frizzy or ethnic hair. The above-described reaction mechanism for cleaving the disulfide bonds of the keratin to form —SH groups without subsequent oxidation is also utilized in depilation, the chemical removal of hair from skin. After the keratin-reducing substance(s) act(s) on the skin, for example on the legs, the hair is easily scraped off without the need for shaving, which irritates the skin.

Within the meaning of the invention, a compound is understood as keratin-reducing when a solution of 50 mmol of this compound in 100 g water at 20° C. and a pH of 8 (set with 2-aminoethanol) cleaves disulfide bonds of the keratin to form —SH groups.

Customary keratin-reducing substances are selected from thioglycolic acid and the salts thereof, and/or thiolactic acid and the salts thereof, and/or cysteine and/or acetylcysteine and/or cysteamine and the salts thereof, and also mixtures thereof. These substances have a distinct, very unpleasant sulfurous odor.

To develop a satisfactory keratin-reducing effect, the agents according to the invention generally require an alkaline or slightly acidic pH for the application. Optimal results are achieved in particular at pH values in the range of 7 to 9.5. Thus far, ammonia has been the alkalizing agent of choice for setting these pH values. Not only can ammonia be used to set the necessary pH range for the dye formation, but it also provides for swelling of the hair to a greater extent than all other known alkalizing agents. At the same time, ammonia acts as a penetration agent or penetration auxiliary agent, likewise to a greater extent than all other standard alkalizing agents. The application-related advantages associated with the use of ammonia are so numerous that, despite its unpleasant, pungent odor, ammonia is used in many commercially available straightening, wave set, and depilatory agents.

One option in principle for reducing the sulfurous odor involves the addition of specific fragrance substances intended to mask the odor. However, fragrance substances may be unstable under the alkaline storage conditions, so that there is a risk that the fragrances may break down or become structurally changed during storage, which is also reflected in an unpredictable change in the odor. Since such changes are often not perceivable until after several months or even years, the use of new or unknown fragrances is considered to be problematic.

Another option in principle for reducing the sulfurous odor is optimization of the formulation. This involves selection of the carrier components of the formulation in such a way that they ensure optimal retention of the keratin-reducing substances in the formulation, and thus minimize the odor thereof. However, it is likewise known that the formulation, the fatty substances optionally included therein, its emulsifiers and surfactants, and its viscosity have a significant influence on the keratolytic effect. Therefore, in any case an impairment of the keratolytic effect should be avoided in the modification of the formulation.

In particular, the long-lasting minimization of odor over the entire period of use is achieved only with great difficulty. The period of time during which the user is in contact with the wave set, straightening, and depilatory agents extends from the application of the agent to the hair, and the exposure time, until the formulation is washed out. For customary exposure times of 5-60 minutes, preferably 10-30 minutes, the overall process may take up to 75 minutes. Effective masking of the odor of the keratin-reducing substances over this entire period represents a tremendous challenge. There is a need for even greater optimization in particular in this field, and an optimal option for the long-lasting reduction of odor of the keratin-reducing substances is thus far not known from the prior art.

The object of the present invention, therefore, is to provide agents for straightening, permanently shaping, or depilation of hair which have a reduced sulfurous odor. In this regard, the agents should have no loss in their keratin-reducing effect.

In particular, the object of the present invention is to achieve a reduction in the sulfurous odor over the entire period of use. The perception of odor of the keratin-reducing substances should still be effectively minimized, even after a maximum contact for two hours.

In the course of the studies leading to the present invention, it has surprisingly been found that it is possible to effectively minimize the perception of odor of keratin-reducing substances in agents for straightening, permanently shaping, or depilation of keratinous fibers over the entire period of use when, in addition to the at least one keratin-reducing compound and water, a combination of specific fatty alcohols and specific glyceryl fatty acid esters is added to the agents.

Furthermore, other desirable features and characteristics of the present invention will become apparent from the subsequent detailed description of the invention and the appended claims, taken in conjunction with this background of the invention.

### BRIEF SUMMARY OF THE INVENTION

An agent for straightening, permanently shaping, or depilation of keratinous fibers, in particular human hair, including at least one linear alkan-1-ol or alken-1-ol in each case having 20 to 22 carbon atoms, wherein the at least one alken-1-ol has one to four olefinic double bonds, in a total quantity of 1.8 to 10.0% by weight, based on the weight of the agent; at least one glyceryl fatty acid ester of general formula (I),

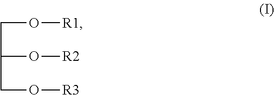

where R1, R2, and R3 independently stand for a hydrogen atom or a group of formula (II),

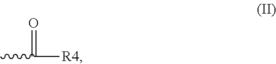

where R4 stands for an unbranched or branched, saturated or unsaturated C₁₁-C₂₇ alkyl group, with the condition that at least one and at most two of the moieties selected from R1, R2, and R3 stand(s) for a group of formula (II); water; and at least one keratin-reducing compound selected from thioglycolic acid and the salts thereof, and/or thiolactic acid and the salts thereof, and/or cysteine and/or acetylcysteine and/or cysteamine and the salts thereof, and also mixtures of these keratin-reducing compounds.

Use of a combination of at least one linear alkan-1-ol or alken-1-ol in each case having 20 to 22 carbon atoms, wherein the at least one alken-1-ol has one to four olefinic double bonds, in a total quantity of 1.8 to 10.0% by weight, based on the weight of the agent; at least one glyceryl fatty acid ester of general formula (I),

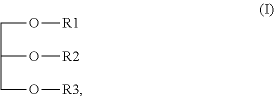

where R1, R2, and R3 independently stand for a hydrogen atom or a group of formula (II),

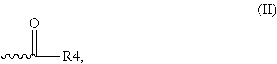

where R4 stands for an unbranched or branched, saturated or unsaturated C₁₁-C₂₇ alkyl group, with the condition that at least one and at most two of the moieties selected from R1, R2, and R3 stand(s) for a group of formula (II); and water in agents for straightening, permanently shaping, or depilation of hair, including at least one keratin-reducing compound selected from thioglycolic acid and the salts thereof, and/or thiolactic acid and the salts thereof, and/or cysteine and/or acetylcysteine and/or cysteamine and the salts thereof, and also mixtures of these keratin-reducing compounds, for reducing the odor of the at least one keratin-reducing compound before, during, and after the straightening, shaping, or depilation operation.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present invention relates to an agent for straightening, permanently shaping, or depilation of keratinous fibers, in particular human hair, including
(a) at least one linear alkan-1-ol or alken-1-ol in each case having 20 to 22 carbon atoms in a total quantity of 1.8 to 10.0% by weight, based on the weight of the agent, wherein the at least one alken-1-ol has one to four olefinic double bonds, and
(b) at least one glyceryl fatty acid ester of general formula (I),

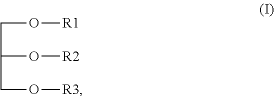

where R1, R2, and R3 independently stand for a hydrogen atom or a group of formula (II),

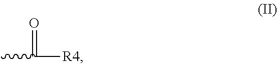

where R4 stands for an unbranched or branched, saturated or unsaturated C₁₁-C₂₇ alkyl group, with the condition that at least one and at most two of the moieties selected from R1, R2, and R3 stand(s) for a group of formula (II),
(c) water, and
(d) at least one keratin-reducing compound selected from thioglycolic acid and the salts thereof, and/or thiolactic acid and the salts thereof, and/or cysteine and/or acetylcysteine and/or cysteamine and the salts thereof, and also mixtures of these keratin-reducing compounds.

Keratin-containing fibers are understood in principle to mean all animal hair, for example wool, horsehair, angora hair, fur, feathers, and products or textiles produced therefrom. However, the keratinous fibers are preferably human hair.

As the first formulation component (a) essential to the invention, the agents according to the invention for straightening, permanently shaping, or depilation of keratin fibers include at least one linear alkan-1-ol or alken-1-ol in each case having 20 to 22 carbon atoms in a total quantity of 1.8 to 10.0% by weight, based on the weight of the agent, wherein the at least one alken-1-ol has one to four olefinic double bonds.

Preferred linear alkan-1-ols and alken-1-ols in each case having 20 to 22 carbon atoms, wherein the at least one alken-1-ol has one to four olefinic double bonds, are selected from the group comprising arachidyl alcohol (eicosan-1-ol), gadoleyl alcohol ((9Z)-eicos-9-en-1-ol), arachidonic alcohol ((5Z,8Z,11Z,14Z)-eicosa-5,8,11,14-tetraen-1-ol), heneicosyl alcohol (heneicosan-1-ol), behenyl alcohol (docosan-1-ol), erucyl alcohol ((13Z)-docos-13-en-1-ol), and brassidyl alcohol ((13E)-docosen-1-ol), and mixtures thereof.

It is therefore preferred when the agents according to the invention include one or more linear alkan-1-ols or alken-1-ols in each case having 20 to 22 carbon atoms from the group comprising arachidyl alcohol (eicosan-1-ol), gadoleyl alcohol ((9Z)-eicos-9-en-1-ol), arachidonic alcohol ((5Z,8Z,11Z,14Z)-eicosa-5, 8,11,14-tetraen-1-ol), heneicosyl alcohol (heneicosan-1-ol), behenyl alcohol (docosan-1-ol), erucyl alcohol ((13Z)-docos-13-en-1-ol), and brassidyl alcohol ((13E)-docosen-1-ol), and mixtures thereof.

Particularly preferred agents according to the invention include one or more linear alkan-1-ols or alken-1-ols in each case having 20 to 22 carbon atoms, wherein the at least one alken-1-ol has one to four olefinic double bonds, preferably at least one alkanol/alkenol of the above-mentioned group, in a total quantity of 1.8 to 10.0% by weight, preferably 2.0 to 8.0% by weight, more preferably 2.1 to 6.0% by weight, in each case based on the weight of the agent.

In another preferred embodiment, an agent according to the invention is therefore characterized in that it includes one or more linear alkan-1-ols or alken-1-ols from the group comprising arachidyl alcohol (eicosan-1-ol), gadoleyl alcohol ((9Z)-eicos-9-en-1-ol), arachidonic alcohol ((5Z,8Z,11Z,14Z)-eicosa-5, 8,11,14-tetraen-1-ol), heneicosyl alcohol (heneicosan-1-ol), behenyl alcohol (docosan-1-ol), erucyl alcohol ((13Z)-docos-13-en-1-ol), and brassidyl alcohol ((13E)-docosen-1-ol) in a total quantity of 1.8 to 10.0% by weight, preferably 2.0 to 8.0% by weight, more preferably 2.1 to 6.0% by weight, in each case based on the weight of the agent.

As the second component essential to the invention, the agent according to the invention includes at least one glyceryl fatty acid ester of general formula (I),

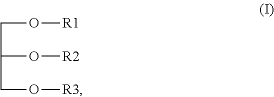

where R1, R2, and R3 independently stand for a hydrogen atom or a group of formula (II),

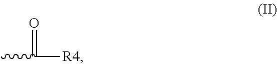

where R4 stands for an unbranched or branched, saturated or unsaturated C₁₁-C₂₇ alkyl group, with the condition that at least one and at most two of the moieties selected from R1, R2, and R3 stand(s) for a group of formula (II).

R4 preferably stands for an unbranched, saturated C₁₁-C₂₇ alkyl group. R4 more preferably stands for an unbranched, saturated C₁₃-C₂₃ alkyl group. R4 particularly preferably stands for an unbranched, saturated C₅-C₁₇ alkyl group.

The glyceryl fatty acid esters (b) in combination with the long-chain fatty alcohols (a) bring about a reduction in the sulfurous odor. The reduction in odor is particularly pronounced when the total quantity of glyceryl fatty acid ester(s) (b) and C20-C22 fatty alcohol(s) (a) are used in certain ratios relative to one another. Therefore, the glyceryl fatty acid esters (b) are also preferably contained in certain quantities in the agent according to the invention.

It is particularly preferred when the agent according to the invention includes one or more glyceryl fatty acid esters (b) in a total quantity of 0.1 to 1.8% by weight, preferably 0.2 to 1.3% by weight, more preferably 0.25 to 0.8% by weight, and particularly preferably 0.3 to 0.6% by weight, based on the total weight of the ready-to-apply agent.

In another particularly preferred embodiment, an agent according to the invention is characterized in that as glyceryl fatty acid ester (b) of general formula (I) it includes at least one compound from the group of formulas (Ia) to (Id).

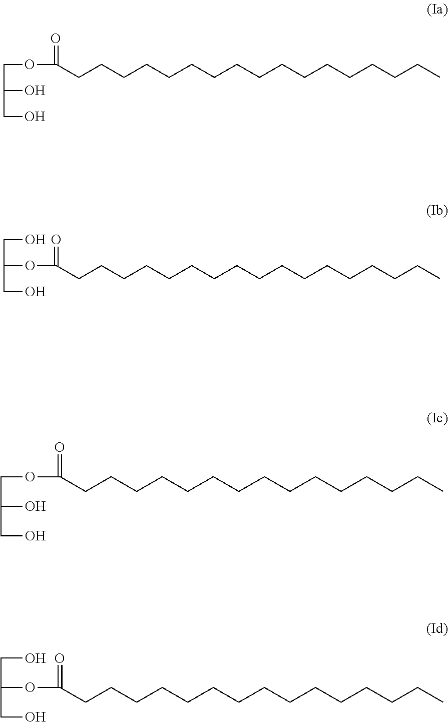

The compounds of formulas (Ia) to (Id) are also known by the names glyceryl monostearate and glyceryl monopalmitate.

In another particularly preferred embodiment, an agent according to the invention is characterized in that as glyceryl fatty acid ester (b) of general formula (I) it includes at least one compound from the group of formulas (Ie) to (Ih).

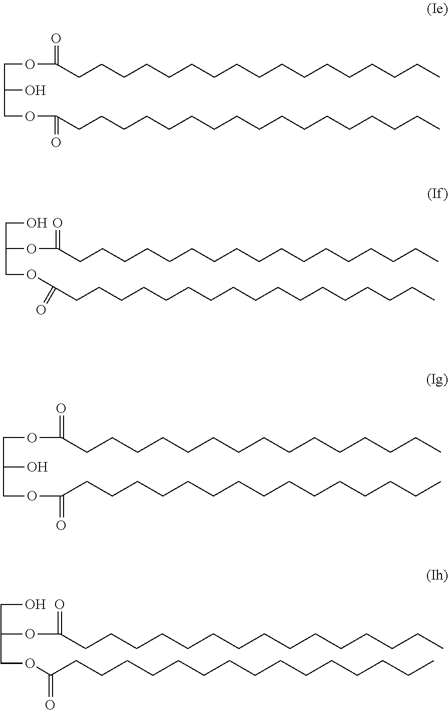

The compounds of formula (Ie) to (Ih) are also known by the names glyceryl distearate and glyceryl dipalmitate.

In one very particularly preferred embodiment, an agent according to the invention is characterized in that as glyceryl fatty acid ester (b) of general formula (I) it includes at least one compound selected from the compounds having formulas (Ia) to (Ih).

In another very particularly preferred embodiment, an agent according to the invention is characterized in that as glyceryl fatty acid ester (b) of general formula (I) it includes at least one compound selected from glyceryl monostearate, glyceryl distearate, glyceryl monopalmitate, and glyceryl dipalmitate, and mixtures thereof.

In another very particularly preferred embodiment, an agent according to the invention is characterized in that as glyceryl fatty acid ester (b) of general formula (I) it includes at least one compound selected from glyceryl monostearate, glyceryl distearate, glyceryl monopalmitate, and glyceryl dipalmitate and mixtures thereof in a total quantity of 0.1 to 1.8% by weight, more preferably 0.2 to 1.3% by weight, more preferably 0.25 to 0.8% by weight, and particularly preferably 0.3 to 0.6% by weight, based on the weight of the agent.

The agents according to the invention include water, preferably in a quantity of 50-80% by weight, particularly preferably 60-75% by weight, in each case based on the weight of the agent.

The agents according to the invention include at least one keratin-reducing compound (d) selected from thioglycolic acid and the salts thereof, and/or thiolactic acid and the salts thereof, and/or cysteine and/or acetylcysteine and/or cysteamine and the salts thereof, and also mixtures of these keratin-reducing compounds. Preferred agents according to the invention are characterized in that they include at least one keratin-reducing compound (d) in a total quantity of 0.1 to 20% by weight, preferably 1 to 15% by weight, particularly preferably 3 to 13% by weight, in each case based on the weight of the agent. Extremely preferred are agents including thioglycolic acid and the salts thereof and/or thiolactic acid and the salts thereof in a total quantity of 5 to 20% by weight, preferably 7 to 15% by weight, particularly preferably 10 to 13% by weight, in each case based on the weight of the agent.

When the above-mentioned keratin-reducing compounds thioglycolic acid, thiolactic acid, cysteine, acetylcysteine, and/or cysteamine are present as the salt, in principle all physiologically acceptable cations are suitable as counterions. In particular, these are metal cations of the physiologically acceptable metals from groups Ia, Ib, IIa, IIb, IIIb, VIa, or VIII of the periodic table of the elements, as well as cationic organic compounds having a quaternized nitrogen atom. The latter are formed, for example, by protonation of primary, secondary, or tertiary organic amines with an acid. Among these, (C₂ to C₆) alkanolammonium ions are preferred. Examples of these cationic organic (C₂ to C₆) alkanolammonium ions are 2-ammonioethanol and 2-trimethylammonioethanol.

According to the invention, the term “(C₂ to C₆) alkanolammonium ion” is understood to mean organic ammonium compounds having two to six carbon atoms which form a carbon framework to which at least one ammonio group (preferably exactly one ammonio group) and at least one hydroxy group (once again preferably exactly one hydroxy group) are bonded. Preferably suited (C₂ to C₆) alkanolammonium ions are 2-ammonioethan-1-ol, 3-ammoniopropan-1-ol, 4-ammoniobutan-1-ol, 5-ammoniopentan-1-ol, 1-ammoniopropan-2-ol, 1-ammoniobutan-2-ol, 1-ammoniopentan-2-ol, 1-ammoniopentan-3-ol, 1-ammoniopentan-4-ol, 3-ammonio-2-methylpropan-1-ol, 1-ammonio-2-methylpropan-2-ol, 3-ammoniopropane-1,2-diol, or 2-ammonio-2-methylpropane-1,3-diol.

If the keratin-reducing compounds bear a chiral center, such as cysteine, any stereoisomers of the stated chiral derivatives are included. The L form is preferred.

In the course of the studies leading to the present invention, it has also been found that the stated object according to the invention may in particular be fully and satisfactorily achieved when the agents according to the invention include further selected formulation components.

Thus, it has been found that the additional presence of certain higher-chain fatty alcohols even further improves the odor result of the compositions according to the invention. Agents preferred according to the invention are therefore characterized in that they additionally include at least one linear saturated alkanol having 12-18 carbon atoms, in particular cetyl alcohol, stearyl alcohol, or lanolin alcohol, or mixtures of these alcohols, which are obtainable in the large-scale industrial hydrogenation of plant and animal fatty acids, as well as mixtures of these alkanols, in particular cetyl alcohol or stearyl alcohol or mixtures thereof, preferably in a total quantity of 0.1 to 10% by weight, particularly preferably 1 to 7% by weight, extremely preferably 2 to 5% by weight, in each case based on the weight of the agent.

It has also been found that the additional presence of certain ethoxylated fatty alcohols having an ethoxylation number of 80 to 120 even further improves the odor result of the compositions according to the invention.

In another very particularly preferred embodiment, an agent for straightening, permanently shaping, or depilation of keratinous fibers is characterized in that as ethoxylated fatty alcohol(s) having an ethoxylation number of 80 to 120 it includes one or more compounds of formula (III),

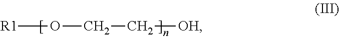

where R1 stands for a saturated or unsaturated, unbranched or branched C₈-C₂₄ alkyl group, preferably for a saturated, unbranched C₁₆ to C₁₈ alkyl group, and
n stands for an integer from 80 to 120, preferably for an integer from 90 to 110, and particularly preferably for the number 100.

According to the invention, fatty alcohols are understood to mean saturated or unsaturated, unbranched or branched C₈-C₂₄ alkyl groups with hydroxyl substitution. Unsaturated fatty alcohols may be monounsaturated or polyunsaturated. For an unsaturated fatty alcohol, its C—C double bond(s) may have the cis or trans configuration.

Preferred fatty alcohols are octan-1-ol (octyl alcohol, capryl alcohol), decan-1-ol (decyl alcohol, capric alcohol), dodecan-1-ol (dodecyl alcohol, lauryl alcohol), tetradecan-1-ol, (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), eicosan-1-ol (eicosyl alcohol, arachidyl alcohol), (9Z)-eicos-9-en-1-ol (gadoleyl alcohol), (5Z,8Z,11Z,14Z)-eicosa-5,8,11,14-tetraen-1-ol (arachidonic alcohol), docosan-1-ol (docosyl alcohol, behenyl alcohol), (13E)-docosen-1-ol (brassidyl alcohol), and (13Z)-docos-13-en-1-ol (erucyl alcohol). Within this group, hexadecan-1-ol (hexadecyl alcohol, cetyl alcohol, palmityl alcohol) and octadecan-1-ol (octadecyl alcohol, stearyl alcohol) are very particularly preferred fatty alcohols.

For forming the optional component, these fatty alcohols are ethoxylated with an ethoxylation number of 80 to 120.

Ethoxylation (also referred to as oxyethylation) is understood to mean the reaction of the fatty alcohols with ethylene oxide (EO). Insertion of 80 to 120 groups of the —CH2—CH2—O— type for each fatty alcohol molecule results in linear polyethers which bear a hydroxy group at one chain end and the C₈-C₂₄ alkyl group of the fatty alcohol at the other chain end.

Preferred ethoxylated fatty alcohols have an ethoxylation number of 90 to 110. It is very particularly preferred when ethoxylated fatty alcohols having an ethoxylation number of 100 are used.

In the course of the studies leading to the present invention, it has been found that the ethoxylation number of the ethoxylated fatty alcohol can surprisingly further improve the capability of the agent for reducing the sulfurous odor. For this reason, it is particularly preferred when one or more ethoxylated fatty alcohols having a specific ethoxylation number is/are used as ethoxylated fatty alcohol(s). It is very particularly preferred when one or more ethoxylated fatty alcohols from the following group is/are used as ethoxylated fatty alcohol:
Dodecan-1-ol (dodecyl alcohol, lauryl alcohol), ethoxylated with 80 EO,
Dodecan-1-ol (dodecyl alcohol, lauryl alcohol), ethoxylated with 81 EO,
Dodecan-1-ol (dodecyl alcohol, lauryl alcohol), ethoxylated with 82 EO,
Dodecan-1-ol (dodecyl alcohol, lauryl alcohol), ethoxylated with 83 EO,
Dodecan-1-ol (dodecyl alcohol, lauryl alcohol), ethoxylated with 84 EO,
Dodecan-1-ol (dodecyl alcohol, lauryl alcohol), ethoxylated with 85 EO,
Dodecan-1-ol (dodecyl alcohol, lauryl alcohol), ethoxylated with 86 EO,
Dodecan-1-ol (dodecyl alcohol, lauryl alcohol), ethoxylated with 87 EO,
Dodecan-1-ol (dodecyl alcohol, lauryl alcohol), ethoxylated with 88 EO,
Dodecan-1-ol (dodecyl alcohol, lauryl alcohol), ethoxylated with 89 EO,
Dodecan-1-ol (dodecyl alcohol, lauryl alcohol), ethoxylated with 90 EO,
Dodecan-1-ol (dodecyl alcohol, lauryl alcohol), ethoxylated with 91 EO,
Dodecan-1-ol (dodecyl alcohol, lauryl alcohol), ethoxylated with 92 EO,
Dodecan-1-ol (dodecyl alcohol, lauryl alcohol), ethoxylated with 93 EO,
Dodecan-1-ol (dodecyl alcohol, lauryl alcohol), ethoxylated with 94 EO,
Dodecan-1-ol (dodecyl alcohol, lauryl alcohol), ethoxylated with 95 EO,
Dodecan-1-ol (dodecyl alcohol, lauryl alcohol), ethoxylated with 96 EO,
Dodecan-1-ol (dodecyl alcohol, lauryl alcohol), ethoxylated with 97 EO,
Dodecan-1-ol (dodecyl alcohol, lauryl alcohol), ethoxylated with 98 EO,
Dodecan-1-ol (dodecyl alcohol, lauryl alcohol), ethoxylated with 99 EO,
Dodecan-1-ol (dodecyl alcohol, lauryl alcohol), ethoxylated with 100 EO,
Dodecan-1-ol (dodecyl alcohol, lauryl alcohol), ethoxylated with 101 EO,
Dodecan-1-ol (dodecyl alcohol, lauryl alcohol), ethoxylated with 102 EO,
Dodecan-1-ol (dodecyl alcohol, lauryl alcohol), ethoxylated with 103 EO,
Dodecan-1-ol (dodecyl alcohol, lauryl alcohol), ethoxylated with 104 EO,
Dodecan-1-ol (dodecyl alcohol, lauryl alcohol), ethoxylated with 105 EO,
Dodecan-1-ol (dodecyl alcohol, lauryl alcohol), ethoxylated with 106 EO,
Dodecan-1-ol (dodecyl alcohol, lauryl alcohol), ethoxylated with 107 EO,
Dodecan-1-ol (dodecyl alcohol, lauryl alcohol), ethoxylated with 108 EO,
Dodecan-1-ol (dodecyl alcohol, lauryl alcohol), ethoxylated with 109 EO,
Dodecan-1-ol (dodecyl alcohol, lauryl alcohol), ethoxylated with 110 EO,
Dodecan-1-ol (dodecyl alcohol, lauryl alcohol), ethoxylated with 111 EO,
Dodecan-1-ol (dodecyl alcohol, lauryl alcohol), ethoxylated with 112 EO,
Dodecan-1-ol (dodecyl alcohol, lauryl alcohol), ethoxylated with 113 EO,
Dodecan-1-ol (dodecyl alcohol, lauryl alcohol), ethoxylated with 114 EO,
Dodecan-1-ol (dodecyl alcohol, lauryl alcohol), ethoxylated with 115 EO,
Dodecan-1-ol (dodecyl alcohol, lauryl alcohol), ethoxylated with 116 EO,
Dodecan-1-ol (dodecyl alcohol, lauryl alcohol), ethoxylated with 117 EO,
Dodecan-1-ol (dodecyl alcohol, lauryl alcohol), ethoxylated with 118 EO,
Dodecan-1-ol (dodecyl alcohol, lauryl alcohol), ethoxylated with 119 EO,
Dodecan-1-ol (dodecyl alcohol, lauryl alcohol), ethoxylated with 120 EO,
Tetradecan-1-ol, (tetradecyl alcohol, myristyl alcohol), ethoxylated with 80 EO,
Tetradecan-1-ol, (tetradecyl alcohol, myristyl alcohol), ethoxylated with 81 EO,
Tetradecan-1-ol, (tetradecyl alcohol, myristyl alcohol), ethoxylated with 82 EO,
Tetradecan-1-ol, (tetradecyl alcohol, myristyl alcohol), ethoxylated with 83 EO,
Tetradecan-1-ol, (tetradecyl alcohol, myristyl alcohol), ethoxylated with 84 EO,
Tetradecan-1-ol, (tetradecyl alcohol, myristyl alcohol), ethoxylated with 85 EO,
Tetradecan-1-ol, (tetradecyl alcohol, myristyl alcohol), ethoxylated with 86 EO,
Tetradecan-1-ol, (tetradecyl alcohol, myristyl alcohol), ethoxylated with 87 EO,
Tetradecan-1-ol, (tetradecyl alcohol, myristyl alcohol), ethoxylated with 88 EO,
Tetradecan-1-ol, (tetradecyl alcohol, myristyl alcohol), ethoxylated with 89 EO,
Tetradecan-1-ol, (tetradecyl alcohol, myristyl alcohol), ethoxylated with 90 EO,
Tetradecan-1-ol, (tetradecyl alcohol, myristyl alcohol), ethoxylated with 91 EO,
Tetradecan-1-ol, (tetradecyl alcohol, myristyl alcohol), ethoxylated with 92 EO,
Tetradecan-1-ol, (tetradecyl alcohol, myristyl alcohol), ethoxylated with 93 EO,
Tetradecan-1-ol, (tetradecyl alcohol, myristyl alcohol), ethoxylated with 94 EO,
Tetradecan-1-ol, (tetradecyl alcohol, myristyl alcohol), ethoxylated with 95 EO,
Tetradecan-1-ol, (tetradecyl alcohol, myristyl alcohol), ethoxylated with 96 EO,
Tetradecan-1-ol, (tetradecyl alcohol, myristyl alcohol), ethoxylated with 97 EO,
Tetradecan-1-ol, (tetradecyl alcohol, myristyl alcohol), ethoxylated with 98 EO,
Tetradecan-1-ol, (tetradecyl alcohol, myristyl alcohol), ethoxylated with 99 EO,
Tetradecan-1-ol, (tetradecyl alcohol, myristyl alcohol), ethoxylated with 100 EO,
Tetradecan-1-ol, (tetradecyl alcohol, myristyl alcohol), ethoxylated with 101 EO,
Tetradecan-1-ol, (tetradecyl alcohol, myristyl alcohol), ethoxylated with 102 EO,
Tetradecan-1-ol, (tetradecyl alcohol, myristyl alcohol), ethoxylated with 103 EO,
Tetradecan-1-ol, (tetradecyl alcohol, myristyl alcohol), ethoxylated with 104 EO,
Tetradecan-1-ol, (tetradecyl alcohol, myristyl alcohol), ethoxylated with 105 EO,
Tetradecan-1-ol, (tetradecyl alcohol, myristyl alcohol), ethoxylated with 106 EO,
Tetradecan-1-ol, (tetradecyl alcohol, myristyl alcohol), ethoxylated with 107 EO,
Tetradecan-1-ol, (tetradecyl alcohol, myristyl alcohol), ethoxylated with 108 EO,
Tetradecan-1-ol, (tetradecyl alcohol, myristyl alcohol), ethoxylated with 109 EO,
Tetradecan-1-ol, (tetradecyl alcohol, myristyl alcohol), ethoxylated with 110 EO,
Tetradecan-1-ol, (tetradecyl alcohol, myristyl alcohol), ethoxylated with 111 EO,
Tetradecan-1-ol, (tetradecyl alcohol, myristyl alcohol), ethoxylated with 112 EO,
Tetradecan-1-ol, (tetradecyl alcohol, myristyl alcohol), ethoxylated with 113 EO,
Tetradecan-1-ol, (tetradecyl alcohol, myristyl alcohol), ethoxylated with 114 EO,
Tetradecan-1-ol, (tetradecyl alcohol, myristyl alcohol), ethoxylated with 115 EO,
Tetradecan-1-ol, (tetradecyl alcohol, myristyl alcohol), ethoxylated with 116 EO,
Tetradecan-1-ol, (tetradecyl alcohol, myristyl alcohol), ethoxylated with 117 EO,
Tetradecan-1-ol, (tetradecyl alcohol, myristyl alcohol), ethoxylated with 118 EO,
Tetradecan-1-ol, (tetradecyl alcohol, myristyl alcohol), ethoxylated with 119 EO,
Tetradecan-1-ol, (tetradecyl alcohol, myristyl alcohol), ethoxylated with 120 EO,
Hexadecan-1-ol (hexadecyl alcohol, cetyl alcohol, palmityl alcohol), ethoxylated with 80 EO,
Hexadecan-1-ol (hexadecyl alcohol, cetyl alcohol, palmityl alcohol), ethoxylated with 81 EO,
Hexadecan-1-ol (hexadecyl alcohol, cetyl alcohol, palmityl alcohol), ethoxylated with 82 EO,
Hexadecan-1-ol (hexadecyl alcohol, cetyl alcohol, palmityl alcohol), ethoxylated with 83 EO,
Hexadecan-1-ol (hexadecyl alcohol, cetyl alcohol, palmityl alcohol), ethoxylated with 84 EO,
Hexadecan-1-ol (hexadecyl alcohol, cetyl alcohol, palmityl alcohol), ethoxylated with 85 EO,
Hexadecan-1-ol (hexadecyl alcohol, cetyl alcohol, palmityl alcohol), ethoxylated with 86 EO,
Hexadecan-1-ol (hexadecyl alcohol, cetyl alcohol, palmityl alcohol), ethoxylated with 87 EO,
Hexadecan-1-ol (hexadecyl alcohol, cetyl alcohol, palmityl alcohol), ethoxylated with 88 EO,
Hexadecan-1-ol (hexadecyl alcohol, cetyl alcohol, palmityl alcohol), ethoxylated with 89 EO,
Hexadecan-1-ol (hexadecyl alcohol, cetyl alcohol, palmityl alcohol), ethoxylated with 90 EO,
Hexadecan-1-ol (hexadecyl alcohol, cetyl alcohol, palmityl alcohol), ethoxylated with 91 EO,
Hexadecan-1-ol (hexadecyl alcohol, cetyl alcohol, palmityl alcohol), ethoxylated with 92 EO,
Hexadecan-1-ol (hexadecyl alcohol, cetyl alcohol, palmityl alcohol), ethoxylated with 93 EO,
Hexadecan-1-ol (hexadecyl alcohol, cetyl alcohol, palmityl alcohol), ethoxylated with 94 EO,
Hexadecan-1-ol (hexadecyl alcohol, cetyl alcohol, palmityl alcohol), ethoxylated with 95 EO,
Hexadecan-1-ol (hexadecyl alcohol, cetyl alcohol, palmityl alcohol), ethoxylated with 96 EO,
Hexadecan-1-ol (hexadecyl alcohol, cetyl alcohol, palmityl alcohol), ethoxylated with 97 EO,
Hexadecan-1-ol (hexadecyl alcohol, cetyl alcohol, palmityl alcohol), ethoxylated with 98 EO,
Hexadecan-1-ol (hexadecyl alcohol, cetyl alcohol, palmityl alcohol), ethoxylated with 99 EO,
Hexadecan-1-ol (hexadecyl alcohol, cetyl alcohol, palmityl alcohol), ethoxylated with 100 EO,
Hexadecan-1-ol (hexadecyl alcohol, cetyl alcohol, palmityl alcohol), ethoxylated with 101 EO,
Hexadecan-1-ol (hexadecyl alcohol, cetyl alcohol, palmityl alcohol), ethoxylated with 102 EO,
Hexadecan-1-ol (hexadecyl alcohol, cetyl alcohol, palmityl alcohol), ethoxylated with 103 ED,
Hexadecan-1-ol (hexadecyl alcohol, cetyl alcohol, palmityl alcohol), ethoxylated with 104 EO,
Hexadecan-1-ol (hexadecyl alcohol, cetyl alcohol, palmityl alcohol), ethoxylated with 105 EO,
Hexadecan-1-ol (hexadecyl alcohol, cetyl alcohol, palmityl alcohol), ethoxylated with 106 EO,
Hexadecan-1-ol (hexadecyl alcohol, cetyl alcohol, palmityl alcohol), ethoxylated with 107 EO,
Hexadecan-1-ol (hexadecyl alcohol, cetyl alcohol, palmityl alcohol), ethoxylated with 108 ED,
Hexadecan-1-ol (hexadecyl alcohol, cetyl alcohol, palmityl alcohol), ethoxylated with 109 EO,
Hexadecan-1-ol (hexadecyl alcohol, cetyl alcohol, palmityl alcohol), ethoxylated with 110 EO,
Hexadecan-1-ol (hexadecyl alcohol, cetyl alcohol, palmityl alcohol), ethoxylated with 111 EO,
Hexadecan-1-ol (hexadecyl alcohol, cetyl alcohol, palmityl alcohol), ethoxylated with 112 EO,
Hexadecan-1-ol (hexadecyl alcohol, cetyl alcohol, palmityl alcohol), ethoxylated with 113 EO,
Hexadecan-1-ol (hexadecyl alcohol, cetyl alcohol, palmityl alcohol), ethoxylated with 114 EO,
Hexadecan-1-ol (hexadecyl alcohol, cetyl alcohol, palmityl alcohol), ethoxylated with 115 EO,
Hexadecan-1-ol (hexadecyl alcohol, cetyl alcohol, palmityl alcohol), ethoxylated with 116 EO,
Hexadecan-1-ol (hexadecyl alcohol, cetyl alcohol, palmityl alcohol), ethoxylated with 117 EO,
Hexadecan-1-ol (hexadecyl alcohol, cetyl alcohol, palmityl alcohol), ethoxylated with 118 EO,
Hexadecan-1-ol (hexadecyl alcohol, cetyl alcohol, palmityl alcohol), ethoxylated with 119 EO,
Hexadecan-1-ol (hexadecyl alcohol, cetyl alcohol, palmityl alcohol), ethoxylated with 120 EO,
Octadecan-1-ol (octadecyl alcohol, stearyl alcohol), ethoxylated with 80 EO,
Octadecan-1-ol (octadecyl alcohol, stearyl alcohol), ethoxylated with 81 EO,
Octadecan-1-ol (octadecyl alcohol, stearyl alcohol), ethoxylated with 82 EO,
Octadecan-1-ol (octadecyl alcohol, stearyl alcohol), ethoxylated with 83 EO,
Octadecan-1-ol (octadecyl alcohol, stearyl alcohol), ethoxylated with 84 EO,
Octadecan-1-ol (octadecyl alcohol, stearyl alcohol), ethoxylated with 85 EO,
Octadecan-1-ol (octadecyl alcohol, stearyl alcohol), ethoxylated with 86 EO,
Octadecan-1-ol (octadecyl alcohol, stearyl alcohol), ethoxylated with 87 EO,
Octadecan-1-ol (octadecyl alcohol, stearyl alcohol), ethoxylated with 88 EO,
Octadecan-1-ol (octadecyl alcohol, stearyl alcohol), ethoxylated with 89 EO,
Octadecan-1-ol (octadecyl alcohol, stearyl alcohol), ethoxylated with 90 EO,
Octadecan-1-ol (octadecyl alcohol, stearyl alcohol), ethoxylated with 91 EO,
Octadecan-1-ol (octadecyl alcohol, stearyl alcohol), ethoxylated with 92 EO,
Octadecan-1-ol (octadecyl alcohol, stearyl alcohol), ethoxylated with 93 EO,
Octadecan-1-ol (octadecyl alcohol, stearyl alcohol), ethoxylated with 94 EO,
Octadecan-1-ol (octadecyl alcohol, stearyl alcohol), ethoxylated with 95 EO,
Octadecan-1-ol (octadecyl alcohol, stearyl alcohol), ethoxylated with 96 EO,
Octadecan-1-ol (octadecyl alcohol, stearyl alcohol), ethoxylated with 97 EO,
Octadecan-1-ol (octadecyl alcohol, stearyl alcohol), ethoxylated with 98 EO,
Octadecan-1-ol (octadecyl alcohol, stearyl alcohol), ethoxylated with 99 EO,
Octadecan-1-ol (octadecyl alcohol, stearyl alcohol), ethoxylated with 100 EO,
Octadecan-1-ol (octadecyl alcohol, stearyl alcohol), ethoxylated with 101 EO,
Octadecan-1-ol (octadecyl alcohol, stearyl alcohol), ethoxylated with 102 EO,
Octadecan-1-ol (octadecyl alcohol, stearyl alcohol), ethoxylated with 103 EO,
Octadecan-1-ol (octadecyl alcohol, stearyl alcohol), ethoxylated with 104 EO,
Octadecan-1-ol (octadecyl alcohol, stearyl alcohol), ethoxylated with 105 EO,
Octadecan-1-ol (octadecyl alcohol, stearyl alcohol), ethoxylated with 106 EO,
Octadecan-1-ol (octadecyl alcohol, stearyl alcohol), ethoxylated with 107 EO,
Octadecan-1-ol (octadecyl alcohol, stearyl alcohol), ethoxylated with 108 EO,
Octadecan-1-ol (octadecyl alcohol, stearyl alcohol), ethoxylated with 109 EO,
Octadecan-1-ol (octadecyl alcohol, stearyl alcohol), ethoxylated with 110 EO,
Octadecan-1-ol (octadecyl alcohol, stearyl alcohol), ethoxylated with 111 EO,
Octadecan-1-ol (octadecyl alcohol, stearyl alcohol), ethoxylated with 112 EO,
Octadecan-1-ol (octadecyl alcohol, stearyl alcohol), ethoxylated with 113 EO,
Octadecan-1-ol (octadecyl alcohol, stearyl alcohol), ethoxylated with 114 EO,
Octadecan-1-ol (octadecyl alcohol, stearyl alcohol), ethoxylated with 115 EO,
Octadecan-1-ol (octadecyl alcohol, stearyl alcohol), ethoxylated with 116 EO,
Octadecan-1-ol (octadecyl alcohol, stearyl alcohol), ethoxylated with 117 EO,
Octadecan-1-ol (octadecyl alcohol, stearyl alcohol), ethoxylated with 118 EO,
Octadecan-1-ol (octadecyl alcohol, stearyl alcohol), ethoxylated with 119 EO,
Octadecan-1-ol (octadecyl alcohol, stearyl alcohol), ethoxylated with 120 EO,
(9Z)-Octadec-9-en-1-ol (oleyl alcohol), ethoxylated with 80 EO,
(9Z)-Octadec-9-en-1-ol (oleyl alcohol), ethoxylated with 81 EO,
(9Z)-Octadec-9-en-1-ol (oleyl alcohol), ethoxylated with 82 EO,
(9Z)-Octadec-9-en-1-ol (oleyl alcohol), ethoxylated with 83 EO,
(9Z)-Octadec-9-en-1-ol (oleyl alcohol), ethoxylated with 84 EO,
(9Z)-Octadec-9-en-1-ol (oleyl alcohol), ethoxylated with 85 EO,
(9Z)-Octadec-9-en-1-ol (oleyl alcohol), ethoxylated with 86 EO,
(9Z)-Octadec-9-en-1-ol (oleyl alcohol), ethoxylated with 87 EO,
(9Z)-Octadec-9-en-1-ol (oleyl alcohol), ethoxylated with 88 EO,
(9Z)-Octadec-9-en-1-ol (oleyl alcohol), ethoxylated with 89 EO,
(9Z)-Octadec-9-en-1-ol (oleyl alcohol), ethoxylated with 90 EO,
(9Z)-Octadec-9-en-1-ol (oleyl alcohol), ethoxylated with 91 EO,
(9Z)-Octadec-9-en-1-ol (oleyl alcohol), ethoxylated with 92 EO,
(9Z)-Octadec-9-en-1-ol (oleyl alcohol), ethoxylated with 93 EO,
(9Z)-Octadec-9-en-1-ol (oleyl alcohol), ethoxylated with 94 EO,
(9Z)-Octadec-9-en-1-ol (oleyl alcohol), ethoxylated with 95 EO,
(9Z)-Octadec-9-en-1-ol (oleyl alcohol), ethoxylated with 96 EO,
(9Z)-Octadec-9-en-1-ol (oleyl alcohol), ethoxylated with 97 EO,
(9Z)-Octadec-9-en-1-ol (oleyl alcohol), ethoxylated with 98 EO,
(9Z)-Octadec-9-en-1-ol (oleyl alcohol), ethoxylated with 99 EO,
(9Z)-Octadec-9-en-1-ol (oleyl alcohol), ethoxylated with 100 EO,
(9Z)-Octadec-9-en-1-ol (oleyl alcohol), ethoxylated with 101 EO,
(9Z)-Octadec-9-en-1-ol (oleyl alcohol), ethoxylated with 102 EO,
(9Z)-Octadec-9-en-1-ol (oleyl alcohol), ethoxylated with 103 EO,
(9Z)-Octadec-9-en-1-ol (oleyl alcohol), ethoxylated with 104 EO,
(9Z)-Octadec-9-en-1-ol (oleyl alcohol), ethoxylated with 105 EO,
(9Z)-Octadec-9-en-1-ol (oleyl alcohol), ethoxylated with 106 EO,
(9Z)-Octadec-9-en-1-ol (oleyl alcohol), ethoxylated with 107 EO,
(9Z)-Octadec-9-en-1-ol (oleyl alcohol), ethoxylated with 108 EO,
(9Z)-Octadec-9-en-1-ol (oleyl alcohol), ethoxylated with 109 EO,
(9Z)-Octadec-9-en-1-ol (oleyl alcohol), ethoxylated with 110 EO,
(9Z)-Octadec-9-en-1-ol (oleyl alcohol), ethoxylated with 111 EO,
(9Z)-Octadec-9-en-1-ol (oleyl alcohol), ethoxylated with 112 EO,
(9Z)-Octadec-9-en-1-ol (oleyl alcohol), ethoxylated with 113 EO,
(9Z)-Octadec-9-en-1-ol (oleyl alcohol), ethoxylated with 114 EO,
(9Z)-Octadec-9-en-1-ol (oleyl alcohol), ethoxylated with 115 EO,
(9Z)-Octadec-9-en-1-ol (oleyl alcohol), ethoxylated with 116 EO,
(9Z)-Octadec-9-en-1-ol (oleyl alcohol), ethoxylated with 117 EO,
(9Z)-Octadec-9-en-1-ol (oleyl alcohol), ethoxylated with 118 EO,
(9Z)-Octadec-9-en-1-ol (oleyl alcohol), ethoxylated with 119 EO,
(9Z)-Octadec-9-en-1-ol (oleyl alcohol), ethoxylated with 120 EO,
(9E)-Octadec-9-en-1-ol (elaidyl alcohol), ethoxylated with 80 EO,
(9E)-Octadec-9-en-1-ol (elaidyl alcohol), ethoxylated with 81 EO,
(9E)-Octadec-9-en-1-ol (elaidyl alcohol), ethoxylated with 82 EO,
(9E)-Octadec-9-en-1-ol (elaidyl alcohol), ethoxylated with 83 EO,
(9E)-Octadec-9-en-1-ol (elaidyl alcohol), ethoxylated with 84 EO,
(9E)-Octadec-9-en-1-ol (elaidyl alcohol), ethoxylated with 85 EO,
(9E)-Octadec-9-en-1-ol (elaidyl alcohol), ethoxylated with 86 EO,
(9E)-Octadec-9-en-1-ol (elaidyl alcohol), ethoxylated with 87 EO,
(9E)-Octadec-9-en-1-ol (elaidyl alcohol), ethoxylated with 88 EO,
(9E)-Octadec-9-en-1-ol (elaidyl alcohol), ethoxylated with 89 EO,
(9E)-Octadec-9-en-1-ol (elaidyl alcohol), ethoxylated with 90 EO,
(9E)-Octadec-9-en-1-ol (elaidyl alcohol), ethoxylated with 91 EO,
(9E)-Octadec-9-en-1-ol (elaidyl alcohol), ethoxylated with 92 EO,
(9E)-Octadec-9-en-1-ol (elaidyl alcohol), ethoxylated with 93 EO,
(9E)-Octadec-9-en-1-ol (elaidyl alcohol), ethoxylated with 94 EO,
(9E)-Octadec-9-en-1-ol (elaidyl alcohol), ethoxylated with 95 EO,
(9E)-Octadec-9-en-1-ol (elaidyl alcohol), ethoxylated with 96 EO,
(9E)-Octadec-9-en-1-ol (elaidyl alcohol), ethoxylated with 97 EO,
(9E)-Octadec-9-en-1-ol (elaidyl alcohol), ethoxylated with 98 EO,
(9E)-Octadec-9-en-1-ol (elaidyl alcohol), ethoxylated with 99 EO,
(9E)-Octadec-9-en-1-ol (elaidyl alcohol), ethoxylated with 100 EO,
(9E)-Octadec-9-en-1-ol (elaidyl alcohol), ethoxylated with 101 EO,
(9E)-Octadec-9-en-1-ol (elaidyl alcohol), ethoxylated with 102 EO,
(9E)-Octadec-9-en-1-ol (elaidyl alcohol), ethoxylated with 103 EO,
(9E)-Octadec-9-en-1-ol (elaidyl alcohol), ethoxylated with 104 EO,
(9E)-Octadec-9-en-1-ol (elaidyl alcohol), ethoxylated with 105 EO,
(9E)-Octadec-9-en-1-ol (elaidyl alcohol), ethoxylated with 106 EO,
(9E)-Octadec-9-en-1-ol (elaidyl alcohol), ethoxylated with 107 EO,
(9E)-Octadec-9-en-1-ol (elaidyl alcohol), ethoxylated with 108 EO,
(9E)-Octadec-9-en-1-ol (elaidyl alcohol), ethoxylated with 109 EO,
(9E)-Octadec-9-en-1-ol (elaidyl alcohol), ethoxylated with 110 EO,
(9E)-Octadec-9-en-1-ol (elaidyl alcohol), ethoxylated with 111 EO,
(9E)-Octadec-9-en-1-ol (elaidyl alcohol), ethoxylated with 112 EO,
(9E)-Octadec-9-en-1-ol (elaidyl alcohol), ethoxylated with 113 EO,
(9E)-Octadec-9-en-1-ol (elaidyl alcohol), ethoxylated with 114 EO,
(9E)-Octadec-9-en-1-ol (elaidyl alcohol), ethoxylated with 115 EO,
(9E)-Octadec-9-en-1-ol (elaidyl alcohol), ethoxylated with 116 EO,
(9E)-Octadec-9-en-1-ol (elaidyl alcohol), ethoxylated with 117 EO,
(9E)-Octadec-9-en-1-ol (elaidyl alcohol), ethoxylated with 118 EO,
(9E)-Octadec-9-en-1-ol (elaidyl alcohol), ethoxylated with 119 EO,
(9E)-Octadec-9-en-1-ol (elaidyl alcohol), ethoxylated with 120 EO,
(9Z,12Z)-Octadeca-9,12-dien-1-ol (linoleyl alcohol), ethoxylated with 80 EO,
(9Z,12Z)-Octadeca-9,12-dien-1-ol (linoleyl alcohol), ethoxylated with 81 EO,
(9Z,12Z)-Octadeca-9,12-dien-1-ol (linoleyl alcohol), ethoxylated with 82 EO,
(9Z,12Z)-Octadeca-9,12-dien-1-ol (linoleyl alcohol), ethoxylated with 83 EO,
(9Z,12Z)-Octadeca-9,12-dien-1-ol (linoleyl alcohol), ethoxylated with 84 EO,
(9Z,12Z)-Octadeca-9,12-dien-1-ol (linoleyl alcohol), ethoxylated with 85 EO,
(9Z,12Z)-Octadeca-9,12-dien-1-ol (linoleyl alcohol), ethoxylated with 86 EO,
(9Z,12Z)-Octadeca-9,12-dien-1-ol (linoleyl alcohol), ethoxylated with 87 EO,
(9Z,12Z)-Octadeca-9,12-dien-1-ol (linoleyl alcohol), ethoxylated with 88 EO,
(9Z,12Z)-Octadeca-9,12-dien-1-ol (linoleyl alcohol), ethoxylated with 89 EO,
(9Z,12Z)-Octadeca-9,12-dien-1-ol (linoleyl alcohol), ethoxylated with 90 EO,
(9Z,12Z)-Octadeca-9,12-dien-1-ol (linoleyl alcohol), ethoxylated with 91 EO,
(9Z,12Z)-Octadeca-9,12-dien-1-ol (linoleyl alcohol), ethoxylated with 92 EO,
(9Z,12Z)-Octadeca-9,12-dien-1-ol (linoleyl alcohol), ethoxylated with 93 EO,
(9Z,12Z)-Octadeca-9,12-dien-1-ol (linoleyl alcohol), ethoxylated with 94 EO,
(9Z,12Z)-Octadeca-9,12-dien-1-ol (linoleyl alcohol), ethoxylated with 95 EO,
(9Z,12Z)-Octadeca-9,12-dien-1-ol (linoleyl alcohol), ethoxylated with 96 EO,
(9Z,12Z)-Octadeca-9,12-dien-1-ol (linoleyl alcohol), ethoxylated with 97 EO,
(9Z,12Z)-Octadeca-9,12-dien-1-ol (linoleyl alcohol), ethoxylated with 98 EO,
(9Z,12Z)-Octadeca-9,12-dien-1-ol (linoleyl alcohol), ethoxylated with 99 EO,
(9Z,12Z)-Octadeca-9,12-dien-1-ol (linoleyl alcohol), ethoxylated with 100 EO,
(9Z,12Z)-Octadeca-9,12-dien-1-ol (linoleyl alcohol), ethoxylated with 101 EO,
(9Z,12Z)-Octadeca-9,12-dien-1-ol (linoleyl alcohol), ethoxylated with 102 EO,
(9Z,12Z)-Octadeca-9,12-dien-1-ol (linoleyl alcohol), ethoxylated with 103 EO,
(9Z,12Z)-Octadeca-9,12-dien-1-ol (linoleyl alcohol), ethoxylated with 104 EO,
(9Z,12Z)-Octadeca-9,12-dien-1-ol (linoleyl alcohol), ethoxylated with 105 EO,
(9Z,12Z)-Octadeca-9,12-dien-1-ol (linoleyl alcohol), ethoxylated with 106 EO,
(9Z,12Z)-Octadeca-9,12-dien-1-ol (linoleyl alcohol), ethoxylated with 107 EO,
(9Z,12Z)-Octadeca-9,12-dien-1-ol (linoleyl alcohol), ethoxylated with 108 EO,
(9Z,12Z)-Octadeca-9,12-dien-1-ol (linoleyl alcohol), ethoxylated with 109 EO,
(9Z,12Z)-Octadeca-9,12-dien-1-ol (linoleyl alcohol), ethoxylated with 110 EO,
(9Z,12Z)-Octadeca-9,12-dien-1-ol (linoleyl alcohol), ethoxylated with 111 EO,
(9Z,12Z)-Octadeca-9,12-dien-1-ol (linoleyl alcohol), ethoxylated with 112 EO,
(9Z,12Z)-Octadeca-9,12-dien-1-ol (linoleyl alcohol), ethoxylated with 113 EO,
(9Z,12Z)-Octadeca-9,12-dien-1-ol (linoleyl alcohol), ethoxylated with 114 EO,
(9Z,12Z)-Octadeca-9,12-dien-1-ol (linoleyl alcohol), ethoxylated with 115 EO,
(9Z,12Z)-Octadeca-9,12-dien-1-ol (linoleyl alcohol), ethoxylated with 116 EO,
(9Z,12Z)-Octadeca-9,12-dien-1-ol (linoleyl alcohol), ethoxylated with 117 EO,
(9Z,12Z)-Octadeca-9,12-dien-1-ol (linoleyl alcohol), ethoxylated with 118 EO,
(9Z,12Z)-Octadeca-9,12-dien-1-ol (linoleyl alcohol), ethoxylated with 119 EO,
(9Z,12Z)-Octadeca-9,12-dien-1-ol (linoleyl alcohol), ethoxylated with 120 EO,
Eicosan-1-ol (eicosyl alcohol, arachidyl alcohol), ethoxylated with 80 EO,
Eicosan-1-ol (eicosyl alcohol, arachidyl alcohol), ethoxylated with 81 EO,
Eicosan-1-ol (eicosyl alcohol, arachidyl alcohol), ethoxylated with 82 EO,
Eicosan-1-ol (eicosyl alcohol, arachidyl alcohol), ethoxylated with 83 EO,
Eicosan-1-ol (eicosyl alcohol, arachidyl alcohol), ethoxylated with 84 EO,
Eicosan-1-ol (eicosyl alcohol, arachidyl alcohol), ethoxylated with 85 EO,
Eicosan-1-ol (eicosyl alcohol, arachidyl alcohol), ethoxylated with 86 EO,
Eicosan-1-ol (eicosyl alcohol, arachidyl alcohol), ethoxylated with 87 EO,
Eicosan-1-ol (eicosyl alcohol, arachidyl alcohol), ethoxylated with 88 EO,
Eicosan-1-ol (eicosyl alcohol, arachidyl alcohol), ethoxylated with 89 EO,
Eicosan-1-ol (eicosyl alcohol, arachidyl alcohol), ethoxylated with 90 EO,
Eicosan-1-ol (eicosyl alcohol, arachidyl alcohol), ethoxylated with 91 EO,
Eicosan-1-ol (eicosyl alcohol, arachidyl alcohol), ethoxylated with 92 EO,
Eicosan-1-ol (eicosyl alcohol, arachidyl alcohol), ethoxylated with 93 EO,
Eicosan-1-ol (eicosyl alcohol, arachidyl alcohol), ethoxylated with 94 EO,
Eicosan-1-ol (eicosyl alcohol, arachidyl alcohol), ethoxylated with 95 EO,
Eicosan-1-ol (eicosyl alcohol, arachidyl alcohol), ethoxylated with 96 EO,
Eicosan-1-ol (eicosyl alcohol, arachidyl alcohol), ethoxylated with 97 EO,
Eicosan-1-ol (eicosyl alcohol, arachidyl alcohol), ethoxylated with 98 EO,
Eicosan-1-ol (eicosyl alcohol, arachidyl alcohol), ethoxylated with 99 EO,
Eicosan-1-ol (eicosyl alcohol, arachidyl alcohol), ethoxylated with 100 EO,
Eicosan-1-ol (eicosyl alcohol, arachidyl alcohol), ethoxylated with 101 EO,
Eicosan-1-ol (eicosyl alcohol, arachidyl alcohol), ethoxylated with 102 EO,
Eicosan-1-ol (eicosyl alcohol, arachidyl alcohol), ethoxylated with 103 EO,
Eicosan-1-ol (eicosyl alcohol, arachidyl alcohol), ethoxylated with 104 EO,
Eicosan-1-ol (eicosyl alcohol, arachidyl alcohol), ethoxylated with 105 EO,
Eicosan-1-ol (eicosyl alcohol, arachidyl alcohol), ethoxylated with 106 EO,
Eicosan-1-ol (eicosyl alcohol, arachidyl alcohol), ethoxylated with 107 EO,
Eicosan-1-ol (eicosyl alcohol, arachidyl alcohol), ethoxylated with 108 EO,
Eicosan-1-ol (eicosyl alcohol, arachidyl alcohol), ethoxylated with 109 EO,
Eicosan-1-ol (eicosyl alcohol, arachidyl alcohol), ethoxylated with 110 EO,
Eicosan-1-ol (eicosyl alcohol, arachidyl alcohol), ethoxylated with 111 EO,
Eicosan-1-ol (eicosyl alcohol, arachidyl alcohol), ethoxylated with 112 EO,
Eicosan-1-ol (eicosyl alcohol, arachidyl alcohol), ethoxylated with 113 EO,
Eicosan-1-ol (eicosyl alcohol, arachidyl alcohol), ethoxylated with 114 EO,
Eicosan-1-ol (eicosyl alcohol, arachidyl alcohol), ethoxylated with 115 EO,
Eicosan-1-ol (eicosyl alcohol, arachidyl alcohol), ethoxylated with 116 EO,
Eicosan-1-ol (eicosyl alcohol, arachidyl alcohol), ethoxylated with 117 EO,
Eicosan-1-ol (eicosyl alcohol, arachidyl alcohol), ethoxylated with 118 EO,
Eicosan-1-ol (eicosyl alcohol, arachidyl alcohol), ethoxylated with 119 EO,
Eicosan-1-ol (eicosyl alcohol, arachidyl alcohol), ethoxylated with 120 EO,
Docosan-1-ol (docosyl alcohol, behenyl alcohol), ethoxylated with 80 EO,
Docosan-1-ol (docosyl alcohol, behenyl alcohol), ethoxylated with 81 EO,
Docosan-1-ol (docosyl alcohol, behenyl alcohol), ethoxylated with 82 EO,
Docosan-1-ol (docosyl alcohol, behenyl alcohol), ethoxylated with 83 EO,
Docosan-1-ol (docosyl alcohol, behenyl alcohol), ethoxylated with 84 EO,
Docosan-1-ol (docosyl alcohol, behenyl alcohol), ethoxylated with 85 EO,
Docosan-1-ol (docosyl alcohol, behenyl alcohol), ethoxylated with 86 EO,
Docosan-1-ol (docosyl alcohol, behenyl alcohol), ethoxylated with 87 EO,
Docosan-1-ol (docosyl alcohol, behenyl alcohol), ethoxylated with 88 EO,
Docosan-1-ol (docosyl alcohol, behenyl alcohol), ethoxylated with 89 EO,
Docosan-1-ol (docosyl alcohol, behenyl alcohol), ethoxylated with 90 EO,
Docosan-1-ol (docosyl alcohol, behenyl alcohol), ethoxylated with 91 EO.
Docosan-1-ol (docosyl alcohol, behenyl alcohol), ethoxylated with 92 EO,
Docosan-1-ol (docosyl alcohol, behenyl alcohol), ethoxylated with 93 EO,
Docosan-1-ol (docosyl alcohol, behenyl alcohol), ethoxylated with 94 EO,
Docosan-1-ol (docosyl alcohol, behenyl alcohol), ethoxylated with 95 EO,
Docosan-1-ol (docosyl alcohol, behenyl alcohol), ethoxylated with 96 EO,
Docosan-1-ol (docosyl alcohol, behenyl alcohol), ethoxylated with 97 EO,
Docosan-1-ol (docosyl alcohol, behenyl alcohol), ethoxylated with 98 EO,
Docosan-1-ol (docosyl alcohol, behenyl alcohol), ethoxylated with 99 EO
Docosan-1-ol (docosyl alcohol, behenyl alcohol), ethoxylated with 100 EO,
Docosan-1-ol (docosyl alcohol, behenyl alcohol), ethoxylated with 101 EO,
Docosan-1-ol (docosyl alcohol, behenyl alcohol), ethoxylated with 102 EO,
Docosan-1-ol (docosyl alcohol, behenyl alcohol), ethoxylated with 103 EO,
Docosan-1-ol (docosyl alcohol, behenyl alcohol), ethoxylated with 104 EO
Docosan-1-ol (docosyl alcohol, behenyl alcohol), ethoxylated with 105 EO,
Docosan-1-ol (docosyl alcohol, behenyl alcohol), ethoxylated with 106 EO,
Docosan-1-ol (docosyl alcohol, behenyl alcohol), ethoxylated with 107 EO,
Docosan-1-ol (docosyl alcohol, behenyl alcohol), ethoxylated with 108 EO,
Docosan-1-ol (docosyl alcohol, behenyl alcohol), ethoxylated with 109 EO
Docosan-1-ol (docosyl alcohol, behenyl alcohol), ethoxylated with 110 EO,
Docosan-1-ol (docosyl alcohol, behenyl alcohol), ethoxylated with 111 EO,
Docosan-1-ol (docosyl alcohol, behenyl alcohol), ethoxylated with 112 EO,
Docosan-1-ol (docosyl alcohol, behenyl alcohol), ethoxylated with 113 EO,
Docosan-1-ol (docosyl alcohol, behenyl alcohol), ethoxylated with 114 EO
Docosan-1-ol (docosyl alcohol, behenyl alcohol), ethoxylated with 115 EO,
Docosan-1-ol (docosyl alcohol, behenyl alcohol), ethoxylated with 116 EO
Docosan-1-ol (docosyl alcohol, behenyl alcohol), ethoxylated with 117 EO,
Docosan-1-ol (docosyl alcohol, behenyl alcohol), ethoxylated with 118 EO,
Docosan-1-ol (docosyl alcohol, behenyl alcohol), ethoxylated with 119 EO,
Docosan-1-ol (docosyl alcohol, behenyl alcohol), ethoxylated with 120 EO.

An agent which is very particularly advantageous, and therefore explicitly very particularly preferred for straightening, permanently shaping, or depilation of keratinous fibers, is characterized in that it includes one or more compounds from the following group as ethoxylated fatty alcohol(s):
Hexadecan-1-ol (hexadecyl alcohol, cetyl alcohol, palmityl alcohol), ethoxylated with 90 EO,
Hexadecan-1-ol (hexadecyl alcohol, cetyl alcohol, palmityl alcohol), ethoxylated with 91 EO,
Hexadecan-1-ol (hexadecyl alcohol, cetyl alcohol, palmityl alcohol), ethoxylated with 92 EO,
Hexadecan-1-ol (hexadecyl alcohol, cetyl alcohol, palmityl alcohol), ethoxylated with 93 EO,
Hexadecan-1-ol (hexadecyl alcohol, cetyl alcohol, palmityl alcohol), ethoxylated with 94 EO,
Hexadecan-1-ol (hexadecyl alcohol, cetyl alcohol, palmityl alcohol), ethoxylated with 95 EO,
Hexadecan-1-ol (hexadecyl alcohol, cetyl alcohol, palmityl alcohol), ethoxylated with 96 EO,
Hexadecan-1-ol (hexadecyl alcohol, cetyl alcohol, palmityl alcohol), ethoxylated with 97 EO,
Hexadecan-1-ol (hexadecyl alcohol, cetyl alcohol, palmityl alcohol), ethoxylated with 98 EO,
Hexadecan-1-ol (hexadecyl alcohol, cetyl alcohol, palmityl alcohol), ethoxylated with 99 EO,
Hexadecan-1-ol (hexadecyl alcohol, cetyl alcohol, palmityl alcohol), ethoxylated with 100 EO,
Hexadecan-1-ol (hexadecyl alcohol, cetyl alcohol, palmityl alcohol), ethoxylated with 101 EO,
Hexadecan-1-ol (hexadecyl alcohol, cetyl alcohol, palmityl alcohol), ethoxylated with 102 EO,
Hexadecan-1-ol (hexadecyl alcohol, cetyl alcohol, palmityl alcohol), ethoxylated with 103 EO,
Hexadecan-1-ol (hexadecyl alcohol, cetyl alcohol, palmityl alcohol), ethoxylated with 104 EO,
Hexadecan-1-ol (hexadecyl alcohol, cetyl alcohol, palmityl alcohol), ethoxylated with 105 EO,
Hexadecan-1-ol (hexadecyl alcohol, cetyl alcohol, palmityl alcohol), ethoxylated with 106 EO,
Hexadecan-1-ol (hexadecyl alcohol, cetyl alcohol, palmityl alcohol), ethoxylated with 107 EO,
Hexadecan-1-ol (hexadecyl alcohol, cetyl alcohol, palmityl alcohol), ethoxylated with 108 EO,
Hexadecan-1-ol (hexadecyl alcohol, cetyl alcohol, palmityl alcohol), ethoxylated with 109 EO,
Hexadecan-1-ol (hexadecyl alcohol, cetyl alcohol, palmityl alcohol), ethoxylated with 110 EO,
Octadecan-1-ol (octadecyl alcohol, stearyl alcohol), ethoxylated with 90 EO,
Octadecan-1-ol (octadecyl alcohol, stearyl alcohol), ethoxylated with 91 EO,
Octadecan-1-ol (octadecyl alcohol, stearyl alcohol), ethoxylated with 92 EO,
Octadecan-1-ol (octadecyl alcohol, stearyl alcohol), ethoxylated with 93 EO,
Octadecan-1-ol (octadecyl alcohol, stearyl alcohol), ethoxylated with 94 EO,
Octadecan-1-ol (octadecyl alcohol, stearyl alcohol), ethoxylated with 95 EO,
Octadecan-1-ol (octadecyl alcohol, stearyl alcohol), ethoxylated with 96 EO,
Octadecan-1-ol (octadecyl alcohol, stearyl alcohol), ethoxylated with 97 EO,
Octadecan-1-ol (octadecyl alcohol, stearyl alcohol), ethoxylated with 98 EO,
Octadecan-1-ol (octadecyl alcohol, stearyl alcohol), ethoxylated with 99 EO,
Octadecan-1-ol (octadecyl alcohol, stearyl alcohol), ethoxylated with 100 EO,
Octadecan-1-ol (octadecyl alcohol, stearyl alcohol), ethoxylated with 101 EO,
Octadecan-1-ol (octadecyl alcohol, stearyl alcohol), ethoxylated with 102 EO,
Octadecan-1-ol (octadecyl alcohol, stearyl alcohol), ethoxylated with 103 EO,
Octadecan-1-ol (octadecyl alcohol, stearyl alcohol), ethoxylated with 104 EO,
Octadecan-1-ol (octadecyl alcohol, stearyl alcohol), ethoxylated with 105 EO,
Octadecan-1-ol (octadecyl alcohol, stearyl alcohol), ethoxylated with 106 EO,
Octadecan-1-ol (octadecyl alcohol, stearyl alcohol), ethoxylated with 107 EO,
Octadecan-1-ol (octadecyl alcohol, stearyl alcohol), ethoxylated with 108 EO,
Octadecan-1-ol (octadecyl alcohol, stearyl alcohol), ethoxylated with 109 EO,
Octadecan-1-ol (octadecyl alcohol, stearyl alcohol), ethoxylated with 110 EO.

Particularly preferred ethoxylated fatty alcohols having an ethoxylation number of 80 to 120 are selected from the group
Octadecan-1-ol (octadecyl alcohol, stearyl alcohol), ethoxylated with 90 EO,
Octadecan-1-ol (octadecyl alcohol, stearyl alcohol), ethoxylated with 91 EO,
Octadecan-1-ol (octadecyl alcohol, stearyl alcohol), ethoxylated with 92 EO,
Octadecan-1-ol (octadecyl alcohol, stearyl alcohol), ethoxylated with 93 EO,
Octadecan-1-ol (octadecyl alcohol, stearyl alcohol), ethoxylated with 94 EO,
Octadecan-1-ol (octadecyl alcohol, stearyl alcohol), ethoxylated with 95 EO,
Octadecan-1-ol (octadecyl alcohol, stearyl alcohol), ethoxylated with 96 EO,
Octadecan-1-ol (octadecyl alcohol, stearyl alcohol), ethoxylated with 97 EO,
Octadecan-1-ol (octadecyl alcohol, stearyl alcohol), ethoxylated with 98 EO,
Octadecan-1-ol (octadecyl alcohol, stearyl alcohol), ethoxylated with 99 EO,
Octadecan-1-ol (octadecyl alcohol, stearyl alcohol), ethoxylated with 100 EO,
Octadecan-1-ol (octadecyl alcohol, stearyl alcohol), ethoxylated with 101 EO,
Octadecan-1-ol (octadecyl alcohol, stearyl alcohol), ethoxylated with 102 EO,
Octadecan-1-ol (octadecyl alcohol, stearyl alcohol), ethoxylated with 103 EO,
Octadecan-1-ol (octadecyl alcohol, stearyl alcohol), ethoxylated with 104 EO,
Octadecan-1-ol (octadecyl alcohol, stearyl alcohol), ethoxylated with 105 EO,
Octadecan-1-ol (octadecyl alcohol, stearyl alcohol), ethoxylated with 106 EO,
Octadecan-1-ol (octadecyl alcohol, stearyl alcohol), ethoxylated with 107 EO,
Octadecan-1-ol (octadecyl alcohol, stearyl alcohol), ethoxylated with 108 EO,
Octadecan-1-ol (octadecyl alcohol, stearyl alcohol), ethoxylated with 109 EO,
Octadecan-1-ol (octadecyl alcohol, stearyl alcohol), ethoxylated with 110 EO.

In another preferred embodiment, an agent for straightening, permanently shaping, or depilation of keratinous fibers is therefore characterized in that it includes one or more ethoxylated fatty alcohols having an ethoxylation number of 80 to 120 in a total quantity of 0.2 to 1.5% by weight, preferably 0.3 to 1.2% by weight, more preferably 0.4 to 0.9% by weight, and particularly preferably 0.5 to 0.8% by weight, based on the weight of the agent.

Likewise advantageous with regard to maximum reduction of the sulfurous odor is the presence of at least one further ethoxylated fatty alcohol having an ethoxylation number of 20 to 40, which is preferably included in a total quantity of 0.1 to 5% by weight, particularly preferably 0.5 to 4% by weight, extremely preferably 1.5 to 3% by weight, based on the weight of the agent.

In another likewise preferred embodiment, an agent according to the invention for straightening, permanently shaping, or depilation of keratinous fibers is therefore characterized in that it additionally includes one or more ethoxylated fatty alcohols having an ethoxylation number of 20 to 40. Suitable fatty alcohols having an ethoxylation number of 20 to 40 are
Dodecan-1-ol (dodecyl alcohol, lauryl alcohol), ethoxylated with 20 to 40 EO,
Tetradecan-1-ol, (tetradecyl alcohol, myristyl alcohol), ethoxylated with 20 to 40 EO,
Hexadecan-1-ol (hexadecyl alcohol, cetyl alcohol, palmityl alcohol), ethoxylated with 20 to 40 EO,
Hexadecan-1-ol (hexadecyl alcohol, cetyl alcohol, palmityl alcohol), ethoxylated with 20 to 40 EO,
Octadecan-1-ol (octadecyl alcohol, stearyl alcohol), ethoxylated with 20 to 40 EO,
(9Z)-Octadec-9-en-1-ol (oleyl alcohol), ethoxylated with 20 to 40 EO,
(9E)-Octadec-9-en-1-ol (elaidyl alcohol), ethoxylated with 20 to 40 EO,
(9Z,12Z)-Octadeca-9,12-dien-1-ol (linoleyl alcohol), ethoxylated with 20 to 40 EO,
Eicosan-1-ol (eicosyl alcohol, arachidyl alcohol), ethoxylated with 20 to 40 EO,
Docosan-1-ol (docosyl alcohol, behenyl alcohol), ethoxylated with 20 to 40 EO, and the mixtures thereof.

In one advantageous embodiment, the ethoxylated fatty alcohols having an ethoxylation number of 80 to 120 and the ethoxylated fatty alcohols having an ethoxylation number of 30 are included in certain quantity ranges and in specific weight ratios.

Lastly, in another likewise preferred embodiment, an agent according to the invention for straightening, permanently shaping, or depilation of keratinous fibers is characterized in that it includes the ethoxylated fatty alcohols (III′) having an ethoxylation number of 30 and the ethoxylated fatty alcohols (III) having an ethoxylation number of 80 to 120 in a weight ratio (III′)/(III) of at least 1:1, preferably in a weight ratio of at least 1.5:1, and particularly preferably in a weight ratio of at least 2:1, in each case based on the total quantity of all ethoxylated fatty alcohols (III′) included in the agent according to the invention and the total quantity of all ethoxylated fatty alcohols (III) included in the agent according to the invention.

In another particularly preferred embodiment of the invention, the agents according to the invention include one or more zwitterionic polymers.

Polymers are understood to mean macromolecules having a molecular weight of at least 1000 g/mol, preferably at least 2500 g/mol, particularly preferably at least 5000 g/mol, and which consist of identical repeating organic units. Polymers are produced by polymerization of one monomer type, or by polymerization of various monomer types which are structurally different from one another. If the polymer is produced by polymerization of one monomer type, it is referred to as a homopolymer. If structurally different monomer types are used in the polymerization, the resulting polymer is referred to by those skilled in the art as a copolymer.

The maximum molecular weight of the polymer depends on the degree of polymerization (the number of polymerized monomers), and is determined by the polymerization method. Within the meaning of the present invention, it is preferred when the molecular weight of the zwitterionic polymer is 100,000 to 10⁷ g/mol, preferably 200,000 to 5·10⁶ g/mol, and particularly preferably 500,000 to 1·10⁶ g/mol.

Zwitterionic polymers are understood to mean those polymers which include cationic groups as well as anionic groups in the macromolecule. The cationic groups included in the macromolecule are quaternary ammonium groups. In these quaternary ammonium groups, a positively charged nitrogen atom bears four organic moieties. The anionic groups are —COO⁻ groups or —SO₃⁻ groups.

To achieve a particularly long-lasting and substantial minimization of the sulfurous odor, it is particularly advantageous to also use the zwitterionic polymers in specific quantity ranges.

In another particularly preferred embodiment, an agent for straightening, permanently shaping, or depilation of keratinous fibers is therefore characterized in that it includes one or more zwitterionic polymers in a total quantity of 0.1 to 1.5% by weight, preferably 0.2 to 1.2% by weight, more preferably 0.3 to 0.8% by weight, and particularly preferably 0.4 to 0.6% by weight, based on the weight of the agent.

Preferred zwitterionic polymers are selected from the group comprising
copolymers of dimethyldiallylammonium salts and acrylic acid, for example Polyquaternium-22,
copolymers of dimethyldiallylammonium salts and methacrylic acid,
copolymers of N,N,N-trimethyl-3-[(2-methyl-1-oxo-2-propen-1-yl)amino]-1-propanaminium salts and acrylic acid,
copolymers of N,N,N-trimethyl-3-[(2-methyl-1-oxo-2-propen-1-yl)amino]-1-propanaminium salts and methacrylic acid,
copolymers of N,N,N-trimethyl-2-[(2-methyl-1-oxo-2-propen-1-yl)amino]-1-ethanaminium salts and acrylic acid,
copolymers of N,N,N-trimethyl-2-[(2-methyl-1-oxo-2-propen-1-yl)amino]-1-ethanaminium salts and methacrylic acid,
copolymers of N,N,N-trimethyl-3-[(2-methyl-1-oxo-2-propen-1-yl)amino]-1-propanaminium salts, acrylic acid, and acrylamide, for example Polyquaternium-53,
copolymers of N,N,N-trimethyl-3-[(2-methyl-1-oxo-2-propen-1-yl)amino]-1-propanaminium salts, methacrylic acid, and acrylamide,
copolymers of 1-ethenyl-3-methyl-1H-imidazolium salts, 1-ethenyl-1H-imidazole, 1-ethenyl-2-pyrrolidinone, and methacrylic acid, for example Polyquaternium-86,
copolymers of 1-ethenyl-3-methyl-1H-imidazolium salts, 1-ethenyl-1H-imidazole, 1-ethenyl-2-pyrrolidinone, and acrylic acid.

Mixtures of the above-mentioned preferred zwitterionic polymers may also be included in the agents particularly preferred according to the invention.

Within the group of preferred zwitterionic polymers, very specific zwitterionic polymers are particularly well suited for reducing the sulfurous odor. When these selected polymers are used, not only is the perceived odor of agents preferred according to the invention optimized, but at the same time the wave setting, straightening, or depilation result is improved.

For this reason, in another explicitly very particularly preferred embodiment, an agent for straightening, permanently shaping, or depilation of keratinous fibers is characterized in that it includes one or more zwitterionic polymers having at least one anionic structural unit of formula (IV) and at least one cationic structural unit of formula (V),

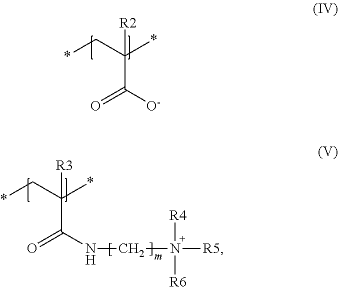

where R2 and R3 independently stand for a hydrogen atom or a methyl group, m stands for an integer from 2 to 6, preferably for the numbers 2 or 3, and the moieties R4, R5, and R6 independently stand for a C₁-C₆ alkyl group, preferably independently stand for a methyl group, an ethyl group, or a propyl group.

One particularly preferred zwitterionic polymer of this type is known by the INCI name Acrylamidopropyltrimonium Chloride/Acrylate Copolymer.

In another likewise particularly preferred embodiment, an agent for straightening, permanently shaping, or depilation of keratinous fibers is therefore characterized in that it includes one or more zwitterionic polymers having at least one anionic structural unit of formula (IV) and at least one cationic structural unit of formula (V) in a total quantity of 0.1 to 1.5% by weight, preferably 0.2 to 1.2% by weight, more preferably 0.3 to 0.8% by weight, and particularly preferably 0.4 to 0.6% by weight, based on the weight of the agent.

In another particularly preferred embodiment, an agent according to the invention for straightening, permanently shaping, or depilation of keratinous fibers is characterized in that it includes one or more zwitterionic polymers having at least one anionic structural unit of formula (VI) and at least one cationic structural unit of formula (VII),

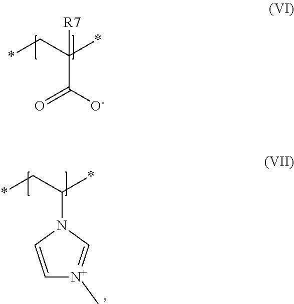

where R7 stands for a hydrogen atom or a methyl group.

In another particularly preferred embodiment, an agent for straightening, permanently shaping, or depilation of keratinous fibers is therefore characterized in that it includes one or more zwitterionic polymers having at least one anionic structural unit of formula (VI) and at least one cationic structural unit of formula (VII) in a total quantity of 0.1 to 1.5% by weight, preferably 0.2 to 1.2% by weight, more preferably 0.3 to 0.8% by weight, and particularly preferably 0.4 to 0.6% by weight, based on the weight of the agent.

In another particularly preferred embodiment, an agent for straightening, permanently shaping, or depilation of keratinous fibers is therefore characterized in that it includes N,N,N-trimethyl-3-[(1-oxo-2-propenyl)amino]-1-propanaminium chloride, polymer with sodium 2-propenoate with INCI Acrylamidopropyltrimonium Chloride/Acrylate Copolymer as zwitterionic polymer in a total quantity of 0.1 to 1.5% by weight, preferably 0.2 to 1.2% by weight, more preferably 0.3 to 0.8% by weight, and particularly preferably 0.4 to 0.6% by weight, based on the weight of the agent.

For setting this pH, the permanent wave, hair straightening, or depilatory agents according to the invention typically include alkalizing agents such as ammonia, and alkali and ammonium carbonates and hydrogen carbonates or organic amines, in particular alkanolamines such as monoethanolamine.

Ammonia is preferably used in the form of its aqueous solution. Such aqueous ammonia solutions may be 10 to 35 percent solutions (calculated in % by weight; 100 g aqueous ammonia solution thus includes 10 to 35 g ammonia). Ammonia is preferably used in the form of a 20 to 30% by weight solution, particularly preferably in the form of a 25% by weight solution. In another particularly preferred embodiment, an agent for straightening, permanently shaping, or depilation of keratinous fibers is therefore characterized in that it includes ammonia in a quantity of 0.25 to 1.75% by weight, preferably 0.4 to 1.4% by weight, more preferably 0.6 to 1.1% by weight, and particularly preferably 0.7 to 0.9% by weight, based on the weight of the agent.

Alkanolamines are primary, secondary, or tertiary amines having a C₂-C₆ alkyl base structure bearing at least one hydroxyl group. 2-Aminoethan-1-ol (monoethanolamine), 2-amino-2-methylpropan-1-ol, 2-amino-2-methyl-propane-1,3-diol, and triethanolamine may be named as examples of alkanolamines.

The fixing of the shaped keratin fibers takes place only due to the influence of an oxidizing agent. Hydrogen peroxide is customarily used for this purpose. In one preferred embodiment, the hydrogen peroxide is used as an aqueous solution. Oxidizing agent preparations preferred according to the invention are characterized in that they include 1.0 to 23.0% by weight, more preferably 2.5 to 21.0% by weight, particularly preferably 4.0 to 20.0% by weight, and very particularly preferably 5.0 to 18.0% by weight hydrogen peroxide (calculated as 100% H₂O₂).

It has proven to be advantageous when the oxidizing agent preparations according to the invention additionally include at least one stabilizer or complexing agent for stabilizing the hydrogen peroxide. Particularly preferred stabilizers are in particular EDTA and EDDS, and phosphonates, in particular 1-hydroxyethane-1,1-diphosphonate (HEDP) and/or ethylenediamine tetramethylene phosphonate (EDTMP) and/or diethylenetriamine pentamethylene phosphonate (DTPMP) or sodium salts thereof.

In addition to the zwitterionic polymers, agents preferred according to the invention may also include anionic polymers. Examples of suitable anionic polymers are those commercially available under the trademark Carbopol® or Rheothik®11-80. The polymers marketed under the INCI name Acrylates Copolymers are also suitable anionic polymers. One preferred commercial product is Aculyn® 33 from Dow Corning (formerly from Rohm & Haas), for example. Further preferred anionic polymers are marketed by Dow Corning (formerly by Rohm & Haas) under the trade name Aculyn® 22, and from AkzoNobel under the trade names Structure® 2001 and Structure® 3001.

Examples of suitable cationic polymers that are also usable are Polyquaternium-24 (commercial product: Quatrisoft® LM 200, for example), Copolymer 845 (manufacturer: ISP), Gaffix® VC 713 (manufacturer: ISP), Gafquat®ASCP 1011, Gafquat®HS 110, Luviquat®8155, and Luviquat®MS 370.

In addition, naturally occurring thickeners as well as nonionic guar gums, for example modified guar gums (Jaguar® HP8, Jaguar® HP60, Jaguar® HP120, Jaguar® DC 293, and Jaguar® HP105, for example) and unmodified guar gums (Jaguar® C, for example) may be used. Further suitable thickeners are the scleroglucan gums or xanthan gums, gums, gum arabic, gum ghatti, karaya gum, gum tragacanth, carrageenan gum, agar-agar, locust bean gum, pectins, alginates, starch fractions, and derivatives such as amylose, amylopectin, and dextrins, and cellulose derivatives such as methylcellulose, carboxyalkyl celluloses, and hydroxyalkyl celluloses.

Further anionic, cationic, or amphoteric surfactants may likewise be included in the agents according to the invention. Preferred anionic surfactants are alkyl sulfates, alkyl ether sulfates, and ether carboxylic acids having 10 to 18 C atoms in the alkyl group and up to 12 glycol ether groups in the molecule. Particularly preferred amphoteric surfactants are N-cocoalkylamino propionate, cocoacylaminoethylamino propionate, and C₁₂-C₁₈ acyl sarcosine.

Examples of additionally included preferred cationic surfactants are ammonium halides, in particular chlorides and bromides, such as alkyltrimethylammonium chlorides, dialkyldimethylammonium chlorides, and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, as well as the imidazolium compounds known by the INCI designations Quaternium-27 and Quaternium-83.

In addition, the agents according to the invention may include further active substances, auxiliary substances, and additives, for example nonionic polymers, silicones, cationic polymers, structurizers, solvents, and mediators, fiber structure-improving active substances, defoamers such as silicones, anti-dandruff active substances, protein hydrolysates, plant oils, for example macadamia nut oil, kukui nut oil, palm oil, amaranth seed oil, peach kernel oil, avocado oil, olive oil, coconut oil, rapeseed oil, sesame oil, jojoba oil, soybean oil, peanut oil, evening primrose oil, and tea tree oil, light protection agents, substances for adjusting the pH, for example common acids, in particular edible acids, vitamins, provitamins, and vitamin precursors, plant extracts, consistency enhancers, waxes, further swelling and penetration substances, pearlescence agents such as ethylene glycol mono- and distearate and PEG-3-distearate, propellants such as propane-butane mixtures, N₂O, dimethyl ether, CO₂, and air, and antioxidants.

Those skilled in the art will select these further substances according to the desired properties of the agents.

The additional active substances and auxiliary substances are preferably included in the agents according to the invention in each case in quantities of 0.0001 to 10% by weight, in particular 0.0005 to 5% by weight, based on the weight of the agent.

Shaping processes on keratin fibers as well as depilation usually proceed in an alkaline or slightly acidic environment. However, to protect the keratin fibers and also the skin to the greatest extent possible, setting a pH that is too high is not desirable. It is therefore preferred when the pH of the agent according to the invention has a value of 5 to 13. Particularly preferred straightening and wave setting agents have a pH in the range of 7 to 9.5. Particularly preferred depilatory agents according to the invention have a pH in the range of 10.5 to 12.7. Within the meaning of the present invention, the stated pH values are values that have been measured at a temperature of 22° C.

Further subject matter of the present invention relates to the use of a combination of
(a) at least one linear alkan-1-ol or alken-1-ol in each case having 20 to 22 carbon atoms, wherein the at least one alken-1-ol has one to four olefinic double bonds, in a total quantity of 1.8 to 10.0% by weight, based on the weight of the agent, and
(b) at least one glyceryl fatty acid ester of general formula (I),

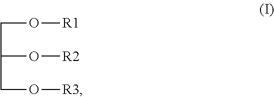

where R1, R2, and R3 independently stand for a hydrogen atom or a group of formula (II),

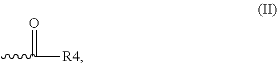

where R4 stands for an unbranched or branched, saturated or unsaturated C₁₁-C₂₇ alkyl group, with the condition that at least one and at most two of the moieties selected from R1, R2, and R3 stand(s) for a group of formula (II), and
(c) water
in agents for straightening, permanently shaping, or depilation of hair, including at least one keratin-reducing compound selected from thioglycolic acid and the salts thereof, and/or thiolactic acid and the salts thereof, and/or cysteine and/or acetylcysteine and/or cysteamine and the salts thereof, and also mixtures of these keratin-reducing compounds, for reducing the odor of the at least one keratin-reducing compound before, during, and after the straightening, shaping, or depilation operation.

Further subject matter of the present invention relates to a method for straightening, permanently shaping, or depilation of keratinous fibers, in which
(i) the fibers are deformed with the aid of mechanical deformation means such as curlers or papillotes before, during, or after step (ii),
(ii) an agent according to the embodiments disclosed herein,
(iii) after an exposure time Z1, preferably after 5-60 minutes, particularly preferably after 10-30 minutes, the fibers are rinsed and optionally dried,
(iv) a fixing agent including at least one oxidizing agent is subsequently applied to the fibers and rinsed off after an exposure time Z2, preferably after 1-30 minutes, particularly preferably after 5-20 minutes.

The statements made with regard to the agents according to the invention also apply mutatis mutandis with regard to further preferred embodiments of the methods and uses according to the invention.

### Examples

The following straightening agents were prepared (all stated quantities are in % by weight):

| | Example | |
|---|---|---|
| | according to | Comparative |
| | the invention | example |
| Cetearyl alcohol (cetyl alcohol and | — | 7.000000 |
| stearyl alcohol in a 1:1 weight ratio) | | |
| Cetyl alcohol | 3.600000 | — |
| Lanette 22 | 2.400000 | 1.000000 |
| 1,2-Propylene glycol | 6.000000 | 1.000000 |
| Eumulgin B 3 | 1.200000 | 0.500000 |
| Cutina GMS-V | 0.600000 | 1.000000 |
| Hydroxyethylcellulose | — | 0.300000 |
| Ammonium thioglycolate (71% by | 9.200000 | 9.200000 |
| weight in water) | | |
| HEDP (60% by weight in | 0.300000 | 0.300000 |
| water) | | |
| Ammonia (25% by weight in | 1.200000 | 1.200000 |
| water) | | |
| Fragrance | 0.700000 | 0.700000 |
| Ammonium hydrogen carbonate | 3.000000 | 3.000000 |
| Paraffinum Liquidum | 2.100000 | — |
| Brij S100-PA | 0.600000 | — |
| Product W 37194 | 1.250000 | — |
| Water, demineralized | ad 100.000000 | ad 100.000000 |

Both straightening agents were smelled by perfumers.

The example according to the invention showed a much fainter sulfurous odor of thioglycolic acid than the comparative example.

List of Raw Materials Used

| Product name | INCI | | Manufacturer |
|---|---|---|---|
| Brij S100-PA | Steareth-100 | | Croda |
| Cutina GMS-V | Glyceryl | Glyceryl | BASF |
| | Stearate | monostearate and | |
| | | glyceryl distearate | |
| | | in a 1:1 weight ratio | |
| Eumulgin B 3 | Ceteareth-30 | | BASF |
| Lanette 22 | Behenyl Alcohol | C18:0 alcohol: | BASF |
| | | 5-15% by weight, | |
| | | C20:0 alcohol: | |
| | | 10-20% by weight, | |
| | | C22:0 alcohol: | |
| | | 70-80% by weight | |
| HEDP, 60% | Etidronic Acid, | | |
| by weight | Aqua (water) | | |
| Product W | Acrylamidopro- | Sodium acrylate, | Bozzetto |
| 37194 | pyltrimonium | trimethyl | GmbH |
| | Chloride/ | ammoniopropyl- | (Stockhausen) |
| | Acrylates | acrylamide | |
| | Copolymer, Aqua | chloride copolymer | |
| | (water) | (19-21% by weight | |
| | | in water) | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for straightening, permanently shaping, or depilation of keratinous fibers, comprising
(a) at least one linear alkan-1-ol or alken-1-ol in each case having 20 to 22 carbon atoms, wherein the at least one alken-1-ol has one to four olefinic double bonds, in a total quantity of 1.8 to 10.0% by weight, based on the weight of the agent, and
(b) at least one glyceryl fatty acid ester of general formula (I), 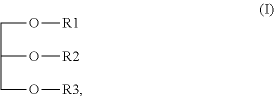
where
R1, R2, and R3 independently stand for a hydrogen atom or a group of formula (II), 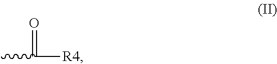
where
R4 stands for an unbranched or branched, saturated or unsaturated C₁₁-C₂₇ alkyl group,
with the condition that at least one and at most two of the moieties selected from R1, R2, and R3 stand(s) for a group of formula (II),
(c) water, and
(d) at least one keratin-reducing compound selected from the group consisting of:
thioglycolic acid and the salts thereof, thiolactic acid and the salts thereof, cysteine and salts thereof, acetylcysteine and salts thereof, cysteamine and the salts thereof, and also mixtures of these keratin-reducing compounds.

2. The agent according to claim 1, wherein the at least one keratin-reducing compound (d) is included in a total quantity of 0.1 to 20% by weight based on the total weight of the agent.

3. The agent according to claim 1, wherein the at least one keratin-reducing compound (d) is included in a total quantity of 1 to 15% by weight based on the total weight of the agent.

4. The agent according to claim 1, wherein the at least one keratin-reducing compound (d) is included in a total quantity of 3 to 13% by weight based on the total weight of the agent.

5. The agent according to claim 1, wherein the at least one linear alkan-1-ol or alken-1-ol in each case having 20 to 22 carbon atoms is selected from the group consisting of arachidyl alcohol (eicosan-1-ol), gadoleyl alcohol ((9Z)-eicos-9-en-1-ol), arachidonic alcohol ((5Z,8Z,11Z,14Z)-eicosa-5, 8,11,14-tetraen-1-ol), heneicosyl alcohol (heneicosan-1-ol), behenyl alcohol (docosan-1-ol), erucyl alcohol ((13Z)-docos-13-en-1-ol), and brassidyl alcohol ((13E)-docosen-1-ol), and mixtures thereof.

6. The agent according to claim 1, wherein the at least one linear alkan-1-ol or alken-1-ol in each case having 20 to 22 carbon atoms is selected from the group of arachidyl alcohol, behenyl alcohol, and mixtures thereof.

7. The agent according to claim 1, wherein the at least one linear alkan-1-ol or alken-1-ol in each case having 20 to 22 carbon atoms comprises 0.5 to 10.0% by weight of the agent.

8. The agent according to claim 1, wherein the at least one linear alkan-1-ol or alken-1-ol in each case having 20 to 22 carbon atoms comprises 1.0 to 8.0 by weight of the agent.

9. The agent according to claim 1, wherein the at least one linear alkan-1-ol or alken-1-ol in each case having 20 to 22 carbon atoms comprises 1.8 to 6.0% by weight of the agent.

10. The agent according to claim 1, wherein the at least one glyceryl fatty acid ester (b) of general formula (I) is selected from the group consisting of glyceryl monostearate, glyceryl distearate, glyceryl monopalmitate, and glyceryl dipalmitate, and mixtures thereof.

11. The agent according to claim 1, wherein the at least one glyceryl fatty acid ester (b) of general formula (I) comprises 0.1 to 1.8% by weight of the agent.

12. The agent according to claim 1, wherein the at least one glyceryl fatty acid ester (b) of general formula (I) comprises 0.2 to 1.3% by weight of the agent.

13. The agent according to claim 1, further comprising at least one linear saturated alkanol having 12-18 carbon atoms.

14. The agent according to claim 13, wherein the at least one linear saturated alkanol is selected from the group consisting of cetyl alcohol, stearyl alcohol, lanolin alcohol, and mixtures of these alcohols.

15. The agent according to claim 1, further comprising cetyl alcohol, stearyl alcohol, or mixtures thereof.

16. The agent according to claim 1, further comprising 0.1 to 10% by weight, based on the weight of the agent, of at least one linear saturated alkanol having 12-18 carbon atoms.

17. The agent according to claim 1, wherein the agent comprises 50-80% by weight water, based on the total weight of the agent.

18. The agent according to claim 1, further comprising at least one ethoxylated fatty alcohol having an ethoxylation number of 80 to 120 which is selected from one or more compounds of formula (III) 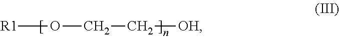
where R1 stands for a saturated or unsaturated, unbranched or branched C₈-C₂₄ alkyl group, preferably for a saturated, unbranched C₁₆ to C₁₈ alkyl group, and n stands for an integer from 80 to 120, preferably for an integer from 90 to 110, and particularly preferably for the number 100.

19. The agent according to claim 18, wherein the in that at least one ethoxylated fatty alcohol comprises 0.2 to 1.5% of the agent.

20. A method for straightening, permanently shaping, or depilation of keratinous fibers, comprising:
(i) the fibers are deformed with the aid of mechanical deformation means such as curlers or papillotes before, during, or after step (ii),
(ii) applying to the fibers an agent according to one of claim 1,
(iii) rinsing the fibers after an exposure time Z1
(iv) optionally drying the fibers
(v) applying a fixing agent to the fibers including at least one oxidizing agent and rinsing off the fixing agent after an exposure time Z2.
